(19) United States
(12) Patent Application Publication
(10) Pub. No.: US 2016/0223513 A1
(43) Pub. Date: Aug. 4, 2016

(54) REPEATABLE AND COMPARABLE INSPECITON OF CONCRETE JOINTS

(71) Applicant: Michael C. Belangie, Freeland, WA (US)

(72) Inventor: Michael C. Belangie, Freeland, WA (US)

(21) Appl. No.: 15/013,081

(22) Filed: Feb. 2, 2016

Related U.S. Application Data
(60) Provisional application No. 62/110,986, filed on Feb. 2, 2015.

(51) Int. Cl. G01N 33/38; G01N 19/10

(52) U.S. Cl. CPC G01N 33/383; G01N 19/10

(57) ABSTRACT

A joint inspection system is described herein that develops and implements a practical, rapid imaging system to detect the presence of contaminants or moisture on the adhesion surfaces of a joint and to identify the presence of micro-fractures, fractures, or anomalies that will affect the integrity of the concrete adjacent to the joint faces (elements that could develop into post-construction spalling). The system utilizes existing technologies to provide an accurate, comprehensive, permanent, reviewable, comparable, location-identifiable imagery of the joint channel and the pavement surfaces immediately adjacent to the channel. The system facilitates the gathering of information (imagery) completely unavailable by current state-of-the-art inspection procedures.

### CROSS-REFERENCE TO RELATED APPLICATIONS

This application claims the benefit of U.S. Provisional Patent Application No. 62/110,986 (Attorney Docket No. BELANGIE001) filed on 2015 Feb. 2, entitled “INSPECTION OF CONCRETE PAVEMENT JOINTS,” which is hereby incorporated by reference.

### BACKGROUND

Concrete highway pavements have a potential service-life in excess of forty years. Many of these pavements are exhibiting major damage within ten years of construction; including significant damage to the concrete panels (slabs), and their supporting substructures. As a pavement deteriorates, user costs increase and driver safety is affected. Reconstruction costs are considerable, frequently exceeding new construction costs. Traffic control for maintenance and reconstruction in high traffic areas are particularly expensive and user delays and safety concerns are significant. Failed joint and joint-sealant systems are a primary cause of these early concrete pavement failures.

In a typical four-lane concrete highway there are approximately 50,000 linear feet of joint, each additional lane adds about 7,000 linear feet of joint. Sealing joints costs about $3 to $4 per linear foot today. Silicone joint-sealant systems are capable of service life in excess of 25 years. In some areas, they are showing up to 50% failure within three years. On average, they reach 50% failure in ten years. There are a few locations with few or no failures (moderate climates with minimal temperature related contraction and expansion (⅛″ unsealed shrinkage cuts also do well in these conditions). Temporary filling or capping (see FIG. 1) lasts from one to three years depending on traffic and costs about $1 per linear foot. Joint resealing (silicone) is more expensive than the initial construction cost; and is typically not done. Joint reconstruction is significantly higher.

Joints are an essential element in concrete pavements. Concrete as it cures shrinks and cracks. Unless controlled, the cracking is random and will result in early, extensive pavement failures. To prevent these failures, an engineered pattern of shrinkage-control cuts is made. However, unsealed, shrinkage-control cuts allow both water to enter the pavement sub-structure and incompressibles (rocks, dirt, sand, and so forth) to enter and lodge in the shrinkage-cut. Entrance of both water and incompressibles are primary causes of concrete pavement damage.

To prevent the entry of water and incompressibles the shrinkage-cut is either filled or widened (joint-cut) and sealed with a designed joint-sealant system. Where the filler or sealant within the filled joint or joint-sealant system has failed (adhesion or cohesion failure), but the concrete joint-channel is relatively intact, water infiltration is the primary cause of pavement failure. Water penetrating into the substructure erodes the base and sub-base. As erosion progresses it undermines the concrete panel. This results, initially, in vertical movement of the panel adjacent to the joint. Concrete is weak in tension and this vertical movement results in the cracking and the eventual break-up of the pavement adjacent to the joint channel.

Temperature change causes concrete panels to expand and contract. This results in the opening and closing of the joint channel. When the filled system, the joint-sealant system, or concrete adjacent to the joint channel (spalling) has failed, the open joint channel can fill with incompressibles. As the temperature rises and the slabs expand, the joint tries to close; but the closure is restricted by the incompressibles. This results in what is called pavement-growth. Pavement-growth can result in pavement blow-ups (where adjacent concrete panels will rise-up vertically forming a wall in the roadway), bridge decks being forced off their supports, and a variety of damage to the affected pavement and adjacent structures.

To prevent concrete pavement damage from unsealed shrinkage control cuts a number of joint-sealant concepts have been tried. Currently there are three basic approaches to joint sealing: no-seal, compression, and formed-in-place.

The no-seal approach is a direct result of the high failure rates of formed-in-place sealants and the high cost of compression seals. Beginning in mid-2000 a number of states adopted a policy of not using a joint-sealant system. These states will use one, or more, of three approaches, as shown in FIG. 1: (A) the shrinkage cut will be left open (B) it will be partially filled with a hot-pour sealant or; (C) it will be filled and capped with a hot-pour sealant. In a filled system the sealant is not expected to adhere (“seal”) to the shrinkage cut walls. This approach assumes high maintenance costs and the possibility of failures similar to those occurring with failed joint-sealant systems. The entities using this approach assume that not having the high initial construction costs will more than offset the costs of higher maintenance and repairing the expected failures. In capping, the sealant in addition to filling the joint is placed over the joint and onto the adjacent pavement. This approach will provide a temporary seal but it is vulnerable to traffic wear. Capping with hot-pours is typically how joints and joint-sealant systems are maintained.

Compression approach systems combine a joint-cut with an engineered, pre-formed sealant that is compressed on installation. Initial costs of compression systems are significantly higher than formed-in-place systems. As a result, their use is minimal. Most entities use formed-in-place sealant systems. This approach typically uses one of two material types either silicones or polymer-modified, asphalt-rubber hot-pours (there are others but their use is nominal). All of these material types involve an engineered joint-channel design (FIG. 2), and clean adhesive joint faces (FIG. 2 (17)) for the sealant to adhere to.

FIG. 2 illustrates a fragmentary section of a typical concrete highway pavement laid down as a continuous length in which cuts are made for accommodating expansion and contraction of adjoining concrete slab sections, here indicated 10 and 11, brought about by reason of changing weather conditions. A usual shrinkage control cut 12, typically one-eighth of an inch in width, is first made to a depth of typically three to four inches transversely across or longitudinally of the concrete as laid to a customary depth of eight to twelve inches. It is common practice to make the cut 12 by use of a diamond saw. This results in a break 13 through the remaining depth of concrete to the sub-grade 16 providing the slabs 10 and 11 as separate but closely adjoining entities. A sealing joint channel 14 is then cut along the length of and extending across the shrinkage control cut 12. Prior to placement of the sealant 15 and the backer rod B the opposing faces 17 of the concrete joint channel are cleaned with high-pressure water-blast, shot-blast, or sandblast, typically high-pressure water-blast.

The major causes of joint-sealant system adhesion failures are inadequate cleaning, moisture on the joint face 17 (the adhesion surface) just prior to sealing, and construction joint-sawing irregularities (saw-cut overlaps and joint-channel widening). Joint-sawing irregularities can affect the placement and stability of the backer rod B and result in the sealant thickness being too great and affecting the sealant's ability to elongate without the increased tension causing adhesion failure.

Cleaning joints is not easy. Joints are narrow and the angle of the water or sand blast can become quite shallow in the middle and lower portions of the adhesion surfaces. It is impossible to thoroughly clean a ⅛″ wide joint with blast-type cleaning methods. Hence the fill and fill and cap approach used in the no-seal method. For formed-in-place joint-sealant systems, it is quite difficult to assure adequate cleaning of a ¼″ wide joint. This is a major problem because 40-50% of formed-in-place joints are ¼″ wide longitudinal joints. Width of transverse joints depends primarily on temperature conditions. Milder climates may use ¼″ joints, cooler climates ⅜″, and cold climates ½″. In very cold conditions or specialized applications, ⅝″ and wider joints may be used. Blast type cleaning systems placed too close to the pavement surface or moved too slowly along the joint can erode the joint corners. Eroded joint corners are undesirable for a number of reasons and cleaning operations are required to minimize this condition. Blast type cleaning operations are typically manual and are both tiring and boring; both of which tend to degrade the adequacy of the cleaning operation.

Moisture on the adhesion surface just prior to sealing can be a major cause of early or later adhesion failures. Moisture may be related to precipitation but is typically due to dew point related condensation. This is particularly the case with water-blast cleaning operations or cooler weather in wet climates. In both circumstances, the sub grade (16) FIG. 2, the lower portions of the joint channel (14), the shrinkage-cut (12), or shrinkage crack (13) may be saturated or have standing water. Regardless, the air in the joint is often stagnant and if high in humidity can result in condensation developing (often quite rapidly) on the joint surfaces with a relatively minor drop in temperature. Because stagnant air in the joint tends to be layered and the more saturated (dense) air is heavier, the condensation is more likely to develop lower in the joint channel (14). Both the rapidity of condensation developing and the location on the lower and/or middle portions of the adhesion surface make its detection with existing procedures unlikely. As a result, moisture related adhesion failure is a major factor in the failure of formed-in-place joint-sealant systems.

Post-construction spalling is the other principal recognized cause of joint-sealant system failure. Post-construction spalling has a number of sources including concrete matrix defects and fractured aggregate, but the primary (largely unrecognized) cause may be “torque-shear” failure. Torque-shear failure is the development of micro-fractures within the concrete adjacent to the joint face because of transverse side-pressure from the concrete joint-saw. The saw operator uses side-pressure to follow deviations in the alignment of the shrinkage-cut. Concrete is weak in tension and the up-lift forces of a joint-saw blade are substantial. Side-pressure in combination with the uplift force exerts shear forces that are resisted by the concrete. If the side pressure is too great during sawing, the concrete will tear-out and spall during construction. To prevent construction spalling the saw operator reduces side-pressure, but the shear forces are often sufficient to create micro-fractures in the concrete adjacent to the joint face.

Micro-fractures will develop into fractures if the concrete matrix containing the micro-fractures is placed in tension. Silicone sealants are elastic materials and when the joint channel opens, the tension on the adhesive channel faces increases and the micro-fractures enlarge. Over-time the cyclical application of tension on the concrete joint channel causes fracturing of the concrete and results in post-construction spalling. Hot-pour (and other) sealants also exert tension on the joint-face when the joint opens. Unlike silicones however, hot-pours stress-relax and the tension on the joint-faces is much less. As a result, construction spalling of hot-pours is about 20% of that exhibited by silicones.

Current inspection procedures are inadequate and are contributing to the extensive failures (adhesion and post-construction spalling) that are occurring in formed-in-place joint-sealant systems. Existing inspection procedures have been in use for over fifty years; and based on the high rate of early failures are very inadequate. Inspection for cleanliness of the joint channel faces is nominal, limited to a minimal number of random insertions of a black cloth into the joint channel and evaluating the cloth surface for change in shade (color). In larger width joints sometimes a finger is used. Another, rarely used procedure involves the insertion of a dental mirror to inspect the surface of the joint channels. All of these methods are labor intensive, have little or no repeatability, and are frequently dependent on the judgment of the inspector. Additional major factors in inadequate inspection procedures are the low-bid process (likelihood of an inferior product), inadequate transportation funding, and the likelihood that inspectors will be involved in multiple projects with significantly different costs. Joint sealing is a comparatively low cost item in comparison to other aspects of a project (or projects) and the higher cost projects tend to monopolize inspector time. All of these factors—inferior construction, an inadequate inspection procedure, and limited inspection time—culminate in high rates of early joint-sealant system failure.

Currently, there is no inspection system in use that is repeatable or comparable. There are no systems in current use that can provide specific, definitive information on the characteristics existing on the joint face. This lack of definitive information has prevented the absolute identification and confirmation of the causes of formed-in-place joint-sealant system failures previously described. There is no system in use: 1) that provides a methodology that permits an evaluation of a section of joint to be repeated or compared with an evaluation of that same section of joint at a later date, 2) that provides a level of detail necessary to determine if moisture is present, or 3) that has the potential to identify micro-fractures surfacing on the joint face.

### BRIEF DESCRIPTION OF THE DRAWINGS

FIG. 1 illustrates three approaches to joint sealing that are common today.

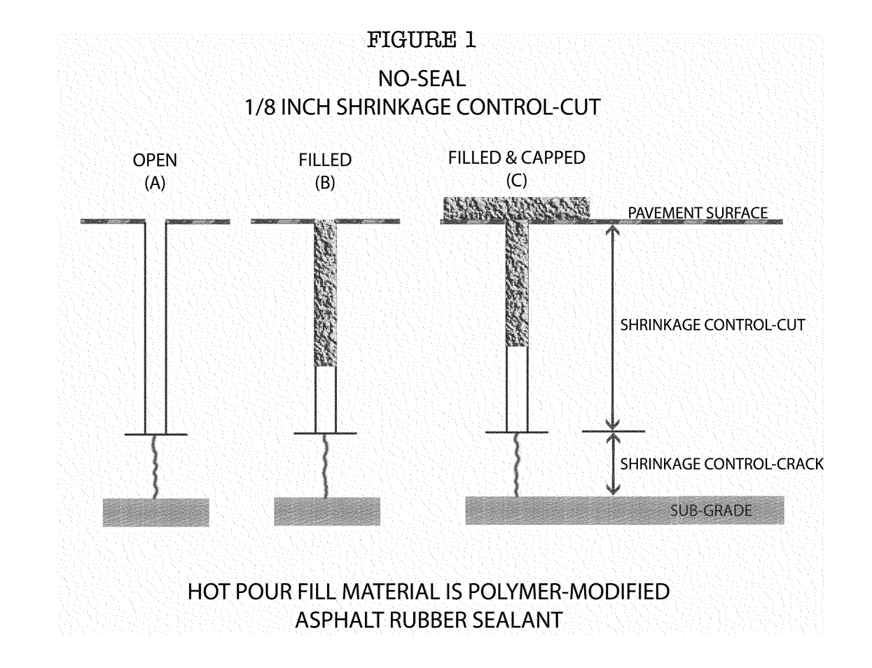

FIG. 2 illustrates a fragmentary section of a typical concrete highway pavement laid down as a continuous length in which cuts are made for accommodating expansion and contraction of adjoining concrete slab sections.

FIG. 3 illustrates components of a vehicle for implementing the joint inspection system, in one embodiment.

FIG. 4 is a block diagram that illustrates components of the joint inspection system, in one embodiment.

FIG. 5 is a flow diagram that illustrates processing of the joint inspection system to inspect a concrete joint, in one embodiment.

FIG. 6 is a flow diagram that illustrates processing of the joint inspection system to compare joint inspection data from two or more subsequent inspections, in one embodiment.

### DETAILED DESCRIPTION

A joint inspection system is described herein that develops and implements a practical, rapid imaging system to detect the presence of contaminants or moisture on the adhesion surfaces of a joint and to identify the presence of micro-fractures, fractures, or anomalies that will affect the integrity of the concrete adjacent to the joint faces (elements that could develop into post-construction spalling). The system utilizes existing technologies to provide an accurate, comprehensive, permanent, reviewable, comparable, location-identifiable imagery of the joint channel and the pavement surfaces immediately adjacent to the channel. The system facilitates the gathering of information (imagery) completely unavailable by current state-of-the-art inspection procedures. In particular, the joint inspection system facilitates:
Utilization of high-definition imagery to identify and evaluate elements appearing on or within the joint channel faces and pavement surfaces immediately adjacent to the joint channel and elements affecting the success or failure of a joint-sealant system.
On-board data storage allows permanent acquisition of high-definition imagery for review by others, documentation, and comparison with subsequent evaluations.
On-board high accuracy GPS permits point-by-point location for identification with subsequent imagery acquired at a later date and location of areas identified for on-site review or sampling.
The development of algorithms for implementing construction improvements including: development of enhanced specifications for improved joint-sealant installation, enhanced quality control, specification compliance, marking of out-of-specification zones, contract documentation, training of both contractors and construction personnel, higher-speed, limited (or more specific) data acquisition, and use of lasers or other technology for topological investigation of the channel faces and sonic (vibration based) or other technology for investigation of the concrete structure supporting the channel faces.

Thus, the joint inspection system greatly improves the quality of joint inspection, and thereby improves the likelihood of proper roadway care that will lead to high longevity.

There are at least five major applications of the joint inspection system: research, specification development, equipment development, training, and construction quality control. Various hardware and software elements are described herein, which can be applied to various embodiments to achieve one or more of these applications. One purpose of the research application is to develop an understanding of the characteristics of joints and to develop methodologies to apply that understanding to construction processes that will result in significantly improved joint and joint-sealant system performance.

In particular, the developed methodologies can result in improved specifications and rapid, effective construction quality control systems to enforce those specifications. This in turn will result in improved joint sawing and cleaning equipment designs, better training of construction and inspection personnel, and documentation to assure compliance with the specification. The various hardware and software elements described herein are not all-inclusive and may or may not be included in various embodiments of the joint inspection system.

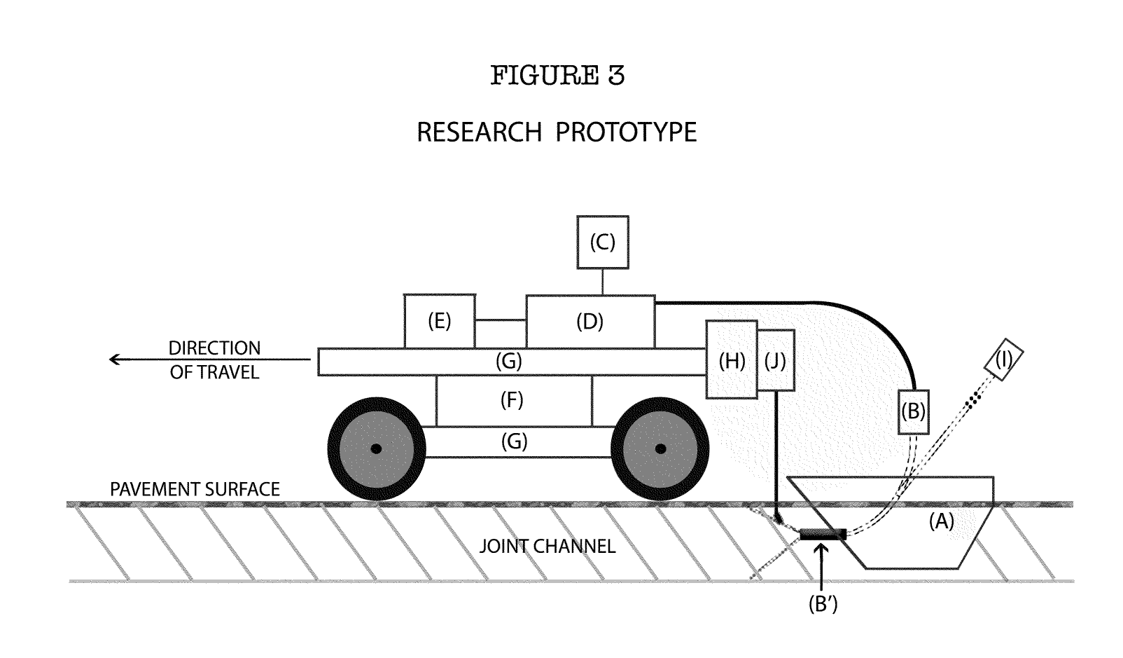

FIG. 3 illustrates components of a vehicle for implementing the joint inspection system, in one embodiment. This embodiment includes a traveler (A), imaging system (B) & (B′), GPS (C), computer (D), data storage (E), power supply (F), carriage (G), marking device (H), endoscope (I), and air pump (J).

The traveler (A) may have exact physical dimensions and on-board components that will vary with application and availability of microminiaturized components. The traveler's basic function is to provide a platform from which continuous, location-integrated imagery (or other data streams) can be acquired. The traveler will, in some embodiments, be suspended from a carriage (G). Depending on the application, and miniaturization, all or some of the image (data) acquisition hardware may be carried on the traveler or allocated between the traveler, the carriage, or some other remote device connected wirelessly or wired.

In one embodiment, the traveler uses two mirrors, one for imaging each joint face. Each mirror is set at approximately 45 degrees to the horizontal plane and 45 degrees to the vertical plane. Each mirror images one joint face and the mirrors have an interior vertical angle of 90 degrees relative to each other. In this configuration, the mirrors reflect light from the bore-scope camera head (B) FIG. 3 (described further herein) onto the imaged face and then reflect the image of the lighted face back to the camera.

This lighting approach may not produce an adequate image for some applications. Other lighting approaches include placing a light source on the traveler, on the carriage, or at some predesigned placement that will provide the desired image qualities (shadow pattern, differential reflection, or other). The topology of the sawed joint face is significantly different from anomalies (slurry, moisture, contaminates) that may be present on the joint face. Different spectrums of light (visible and non-visible) or laser may provide a better image of those or other characteristics (condensate, fractures, micro-fractures) existing on the joint face. These spectrums may be generated either by a source on the traveler or on the camera-head (e.g., appropriate image receiver for the spectrum in use).

The traveler may incorporate one or two wing(s) that extend over and above the pavement surface(s) immediately adjacent to the joint. In some embodiments, the wing(s) may incorporate mirror(s) with a focal distance equivalent to the focal distance of the mirrors mounted on the portion of the traveler within the joint. This permits the camera lens to be mounted in such a way that the image of one joint face and its adjacent pavement surface can be captured at the same time. As an alternative, a second bore-scope or camera system can capture the image from the wing. The wing can identify contaminates that might accidentally enter the joint prior to or during sealing operations that would not be captured by the joint face imaging, and spalling or surface fractures that may result in spalling.

In some embodiments, the traveler also includes the use of sonic or other systems that can reflect, partially penetrate, or penetrate into the concrete matrix supporting the joint face. In this approach, the traveler can function as a receiver or a sender. One purpose of this approach is to provide an image of any micro-fractures or inconsistences in the concrete matrix that would affect the performance of the joint-sealant system.

In some embodiments, the joint inspection system also includes one or more imaging systems (B and B′). Imaging is not limited to the visual spectrum. Imaging can encompass topology oriented (laser-type measuring systems), structural integrity (sonic-type systems, maser), or other approaches to visualizing the integrity of a structure. Regardless of the imaging system in use, the traveler can function as either or both a signal-sender and/or signal-receiver. The location of the imaging system components and their placement on the traveler, the carriage, or remote device, or placement, will be determined by the application and the technology developed or identified and used. Imaging may be processed and stored internally within the imaging device or sent to a remote or on-board (either traveler or carriage, wired or wirelessly) computer.

Agencies vary in their approach to quality control depending on climate, budget, personnel, and other factors. There will be a number of approaches developed (or modified) for construction control of joint cleanliness and moisture free installations using the joint inspection system. These variations may include one or more of the following:
May utilize a bore-scope either suspended above the channel (B) or lowered within the channel (B′).
May dispense with the bore-scope and utilize another type of imaging sensor or combine both types.
May use a screen on the carrier (or other location) to display real-time or stored imagery.
The bore-scope, or sensor system, may take individual readings or video.
May eliminate imagery and depend on pavement marking to identify out of specification areas.
May use varying levels of GPS accuracy or dispense with GPS completely.
May utilize topological sensing systems to discriminate between the texture of a clean joint face and the random surface characteristics of anomalies.
May use visible or non-visible light spectra to differentiate clean and moisture free surfaces from contaminated surfaces.
May utilize sonic, other vibratory type sensors, or other systems to ascertain the integrity of the concrete adjacent to the joint face (e.g., fractures or micro-fractures, fractured aggregate, or other structural anomalies).

In some embodiments, the joint inspection system also includes a carriage (G) for moving the traveler (A) around. The physical dimensions of the carriage and its on-board components will vary with the application and availability of microminiaturized components. The carriage's basic function is to provide a platform from which the traveler can be suspended and moved along the joint channel. Depending on application and miniaturization, all or some of the image (data) acquisition hardware may be carried on the traveler or allocated between the traveler, the carriage, or other remote device (which may or may not be wireless). In some embodiments, a radio controlled model vehicle body is used as the carriage. In other embodiments, the carriage could be a bar mounted or other system that could be carried by the inspector and positioned manually or mounted on a vehicle and positioned either manually or remotely.

In some embodiments, the joint inspection system also includes global positioning system (GPS) hardware (C), or can utilize the GPS hardware of other connected devices (e.g., a smartphone carried by the operator of the joint inspection system). In some applications, the imaging data will be integrated with GPS positioning data. Depending on component size, the GPS receiver system can be mounted on the carriage or other location within the system. Image data is correlated with GPS data so that the location of each image component can be located later; or image-location components taken later can be directly compared with prior data. Construction joint cleanliness/moisture-free quality control systems developed from the high-end devices may not require GPS or may use a much less accurate GPS system. Lower-accuracy GPS may be adequate for contract control and documentation.

In some embodiments, the joint inspection system also includes a computing device (D) and data storage (E). Existing computing and data storage devices are sufficiently small and rugged that they can be located on the traveler, the carriage, or at a remote location. Data transfer can be wired or wireless. One purpose of the computing device is to integrate the input data, place it in data storage, display it to the operator or control the marking system to identify contaminates or other anomalies. Depending on the application, the computing device can control instrumentation input/output functions either independently or in coordination with the operator. In more advanced applications, for instance comparison of the data from a previous inspection with the current inspection; the computing device can be outputting processed information on differences between two or more previous inspections, or providing probabilities of potential failure based on anomalies detected on the joint faces and/or the pavement surface adjacent to the joint.

In some embodiments, the joint inspection system also includes a power supply (F). Depending on the system used, power may be supplied from batteries on the carriage, internal to the devices, or supplied from an external source. Power may be DC or AC depending on device expectations, and may be supplied from a vehicle associated with the carrier or independent of the carrier and utilized by a supplemental system in which the traveler/carrier functions as a receiver for signals (evaluating the integrity of the concrete) resulting from an external sender with an independent power supply or the traveler/carrier can function as a sender to an external receiver.

In some embodiments, the joint inspection system also includes a marking device (H). Marking devices, typically spray paint, may be attached to the carriage and controlled by the onboard computing device or operator to mark areas where the joint face contains anomalies. Depending on the sophistication of the controlling software, the marking system can use different colors to identify the type of contaminate on the joint face (or defect in structural integrity of the concrete adjacent to the joint face).

In some embodiments, the joint inspection system also includes an endoscope (I). A bore-scope similar to a “Teslong” digital video recording endoscope (endoscope) is used in one embodiment. As indicated previously herein, there are multiple possibilities for imaging systems, and the final imaging system for a given variation of the joint inspection system will be selected by researchers or end users.

In some embodiments, the joint inspection system also includes an air pump (J). Construction environments are dusty and the joint channel after cleaning may have residual dust in the air. The use of bursts of low-pressure air directed across the lens is one way of keeping the lens of the camera system or mirrors dust free and this is one purpose of the air pump.

In some embodiments, the joint inspection system may also include other data collection hardware or components. Ambient environmental conditions are a relevant element in many applications. The appropriate instrumentation can be a part of the carriage components, transmitted from a remote device, or otherwise integrated with the system. Instrumentation might include temperature, humidity, and dew point at the surface, in the channel, or both, and other data types.

The joint inspection may include various software applications for controlling the previously described components, for providing a user interface to the operator, for performing various administrative and configuration options, and for generating reports based on the collected data. In some embodiments, the traveler can be configured for either direct imaging or indirect imaging. The primary difference between the two configurations is that with direct imaging the camera or its optics are carried within the joint channel (FIG. 3 (B′)), whereas with indirect imaging the image is reflected from a mirror or prism to camera optics located outside (above) the channel (FIG. 3 (B)).

In some embodiments, with direct imaging a “bore scope” lens, similar to a Teslong digital endoscope camera is positioned on the front of the traveler FIG. 3 (B′) with the optics directed in-line with the channel, or at a preset angle from that alignment; or if the optical head can be actuated, to move in some preset or controlled pattern relative to the in-line alignment. In the indirect-imaging bore scope approach, the camera lens is mounted above the channel FIG. 3 (B) on the traveler and the image is transmitted by fiber optics or other method to the camera system located on the carriage. In some embodiments, it is possible for the carriage to be eliminated and the fiber optic system to be manipulated manually. This approach would be for limited (minimal) inspections and may or may not include the use of the traveler.

An alternate option to the bore-scope approach is to use a micro-miniature camera mounted in the front, or above, the traveler with the same orientation and capabilities as those described for the bore scope. Images in this embodiment are transmitted to a view screen or the computing device for integration with other inputs and then into data storage.

In some embodiments, the traveler can have at least two major imaging variations: high-definition image-acquisition primarily for research; and construction quality control imaging which may be of a lower quality or limited spectrum, with potentially lower GPS accuracy. High-definition image-acquisition includes location-integrated, high-accuracy (GPS), high-definition video taken at a right angle to the channel face or at an angle +/− vertically/horizontally with or without specialized spectrum depending on a particular imaging requirement.

There are at least two imaging approaches for gathering high-definition video: direct view and indirect view. In direct view, the size of the area-of-interest with existing camera optics is limited by the narrowness of the joint channel. As optics and micro-miniaturization develop, direct view imaging may enlarge to encompass the full area-of-interest and direct view may become the imaging system of choice. In some embodiments, the indirect view approach utilizes a mirror or prism, whose length encompasses the full height of the area-of-interest, is set at a 45+x degrees to the vertical and at 45+x degrees to the horizontal, which reflects the image of the full area-of-interest encompassed by the mirror into an optical/camera system placed above the joint channel.

Lighting of the surface to be imaged can be part of the camera system or a separate light source can be mounted on the traveler. Both the intensity and spectrum of the light source can be adapted to a particular imaging requirement.

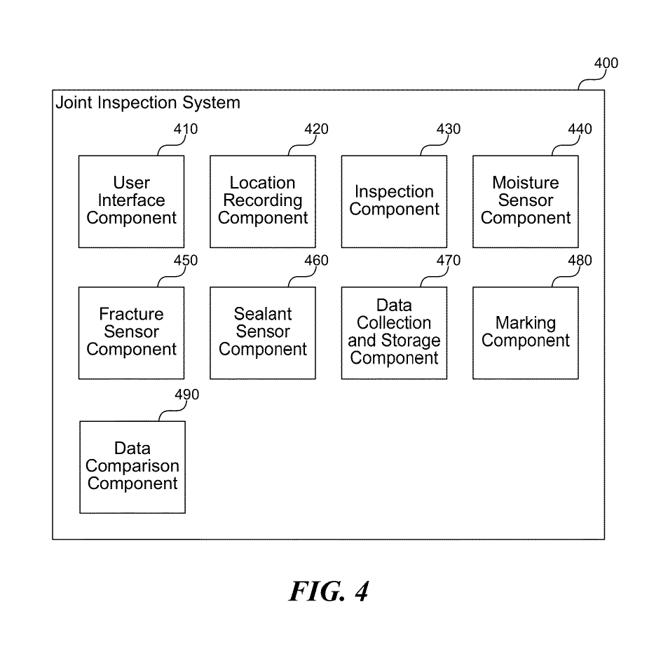

FIG. 4 is a block diagram that illustrates components of the joint inspection system, in one embodiment. The system 400 includes a user interface component 410, a location recording component 420, an inspection component 430, a moisture sensor component 440, a fracture sensor component 450, a sealant sensor component 460, a data collection and storage component 470, a marking component 480, and a data comparison component 490. Only some elements, such as sensors, may be used in a given system. Combinations will vary depending on specific requirements. Each of these components is described in further detail herein.

The user interface component 410 interacts with an operator of the system 400 to receive input and provide output related to a joint inspection. For example, an operator may provide input to the system 400 selecting among multiple types of inspections that can be performed, such as a joint cleanliness inspection versus a joint sealant health inspection. The system 400 may also receive input from the operator specifying a project or job name, location, purpose of the inspection, type of results desired, and so forth. The user interface component 410 may provide various types of output, both during and after inspections. During an inspection, the system 400 may provide instructions to the operator, such as in embodiments where there is manual movement of the carrier vehicle. The system 400 may also display real-time inspection results, such as imagery coming in from one or more cameras or other sensors captured within the joint, as well as offering options such as marking of problem areas. The system 400 may also display comparison data with any earlier inspections performed at the same location, such as to highlight new anomalies or differing conditions from the previous inspection. After an inspection, the system 400 displays results to the operator and may also provide follow up options, such as sending a report to one or more interested parties, setting follow up reminders for remediation of any problems found, and so on.

The location-recording component 420 identifies and captures location information of a location where the joint inspection is taking place. The location information may include high accuracy GPS data, as well as other location identifying data, such as cell tower triangulation data, Wi-Fi and other hotspot data, and so forth. In addition to location, the system 400 may also capture environmental information, such as humidity, temperature, dew point, weather forecast, and the like. This and the other captured location information is associated with the data collected during the inspection, so that later analysis can factor in any relevant environmental factors that may be causes of anomalies or other joint conditions. For example, detection of excess moisture in the joint may have a different meaning in a low humidity environment versus in a high humidity environment. The low humidity environment may point to human factors, such as inadequate drying after joint power washing, whereas the high humidity environment may point to environmental factors, such as high air moisture content and condensation.

The inspection component 430 manages the joint inspection and coordinates the capturing of sensor data and the recording and storage of sensor data for subsequent analysis. The inspection component 430 may guide the operator through the inspection of one or more joints that make up a particular construction project, such as a particular section of roadway. The inspection component 430 may also determine when subsequent inspections will be performed and schedule operators to initiate the inspections. For example, a particular joint sealant product may benefit from annual inspections to ensure correct performance of the product, and the inspection component 430 can manage this schedule to ensure that annual inspections occur and that results can be compared with the results of earlier inspections. The inspection component 430 also manages other optional aspects of each inspection based on the inspection type, such as whether sensor data is captured from both within the joint and the top surface of the joint, whether air or other tools are used to clean the joint during inspection, what light source is used to illuminate the joint for better image capture and/or spectrum analysis, what level of accuracy data is captured, and so forth.

The moisture sensor component 440 senses moisture information within a joint being inspected. A variety of hardware exists for capturing moisture content of the air as well as moisture content of various surfaces or substrates, such as concrete. The system 400 may utilize specialized sensors for capture moisture information or may use more general methods, such as analyzing camera data for the presence of condensation or other signs of moisture. The moisture sensor component 440 and other sensors may be present within a part of the system 400 inserted in the joint and/or in a location outside the joint, such as above the joint. In some cases, a particular project may even embed sensors within the joint for long-term monitoring in accordance with the system 400, which communicate wirelessly or otherwise with the system to relay captured sensor data.

The fracture sensor component 450 detects fractures within a joint that may affect the performance of the joint. For example, the component 450 may detect micro-fractures that could lead to spalling, poor adhesion of joint sealant, or other potential causes of joint failure. A variety of hardware exists for capturing fracture information, such as light-based (e.g., laser, maser, ultraviolet, infrared, or other light spectrum analysis, vibration and sound based, such as sonar, as well as visual techniques, such as can be performed by a camera with a high enough zoom factor to capture microscopic details. The system 400 may also employ an air source as described herein to sufficiently clean the joint of any dust or other debris to allow for increased accuracy of the captured sensor data.

The sealant sensor component 460 is used to inspect previously installed sealant and to assess a present condition of the sealant. Present condition of the sealant may include the sealant's firmness, adhesion to neighboring surfaces, the presence of any cracks/shrinkage/expansion, depth of the sealant from the surface of the roadway, overall depth of the sealant, and so on. The sealant sensor component 460 may use a variety of available off-the-shelf and/or specialized measuring hardware for assessing the condition of the sealant. For example, a durometer might be used to determine the hardness of the sealant, while a camera and digital image analysis might be used to identify cracks and shrinkage of the sealant. Adhesion can be sensed both by visual inspection using a camera and digital image analysis, as well as by manual manipulation, such as a probe that presses on the sealant to determine whether the sealant can be pulled back from the joint surface with a particular amount of force. Early detection of sealant failure can improve the ability of a jurisdiction to schedule resealing or other remediation before more expensive roadway damage has occurred.

The data collection and storage component 470 receives and stores sensor data as well as metadata describing the location and environmental conditions under which sensor data was captured. The system 400 may use a variety of data storage facilities, such as hard drives, flash devices, cloud-based storage services, databases, and other available data storage facilities. The system 400 stores data for longer-term retrieval up to and potentially beyond the expected timing of a subsequent inspection to allow for comparison of data collected from one inspection to the next. The data collection and storage component 470 may maintain a history of each inspection of a particular joint, from construction-time inspection to subsequent maintenance inspections over the years. This data can be used by researchers to note the rate at which joint sealant and other joint conditions progress over time, as well as to identify early signs and symptoms that signaled future joint failure. The data collected and stored by the system 400 is available not just to the operator and vehicle used at the time of capture, but may also be collected and shared in a central location in a manner that researchers or other interested parties can access and analyze data captured in many disparate locations.

The marking component 480 optionally provides a facility to physically mark the joint at locations where one or more joint issues are detected by the system 400. Marking may include paint, chalk, or other types of physical marking of the area that can be detected by workers later that are tasked with performing remediation of any joint issues. Marking may include color coding or other types of distinguishing between multiple types of joint issue, so that the coding of the marking describes to a worker reading the marking what type of issue was detected at that location. For example, red paint might indicate sealant adhesion failure, whereas yellow paint might indicate large debris within the joint. Marking may also be performed by electronic or other non-physical methods, such as in locations where GPS data is sufficiently accurate to tag a particular location in an electronic data store as having a joint issue. Workers carrying electronic devices with this data can then return to the location and perform remediation at the right place even though the location may not be physically marked.

The data comparison component 490 provides analysis of multiple captures of joint data separated in time to identify changes in conditions of the joint between a first inspection and a second inspection of the joint. The data comparison component 490 may output a report to the operator detailing changes in the joint since the last inspection, differences in conditions during the first inspection and the second inspection, and so forth. The data comparison component 490 may also provide predictive output, such as a prediction of likely types of failure of the joint based on the observed conditions and the rate of change of those conditions between the first and second inspections. For example, the system 400 may predict joint adhesion failure based on a change in size (e.g., shrinkage) of the joint sealant between one inspection and the next, such that the operator or the system 400 can schedule resealing of the joint before the adhesion fails and allows more expensive damage to the joint.

Although roadways and pavement are described herein, the system 400 may also be applied to other environments that utilize joints to manage expansion and contraction of concrete and other materials. For example, concrete buildings and other structures also utilize expansion joints and sealants to manage those joints in a manner that can benefit by application of the joint inspection system 400 to monitor joint conditions over time.

The computing device on which the system is implemented may include a central processing unit, memory, input devices (e.g., keyboard and pointing devices), output devices (e.g., display devices), and storage devices (e.g., disk drives or other non-volatile storage media). The memory and storage devices are computer-readable storage media that may be encoded with computer-executable instructions (e.g., software) that implement or enable the system. In addition, the data structures and message structures may be stored on computer-readable storage media. Any computer-readable media claimed herein include only those media falling within statutorily patentable categories. The system may also include one or more communication links over which data can be transmitted. Various communication links may be used, such as the Internet, a local area network, a wide area network, a point-to-point dial-up connection, a cell phone network, and so on.

Embodiments of the system may be implemented in various operating environments that include personal computers, server computers, handheld or laptop devices, multiprocessor systems, microprocessor-based systems, programmable consumer electronics, digital cameras, network PCs, minicomputers, mainframe computers, distributed computing environments that include any of the above systems or devices, set top boxes, systems on a chip (SOCs), and so on. The computer systems may be cell phones, personal digital assistants, smart phones, personal computers, tablet computers, programmable consumer electronics, digital cameras, and so on.

The system may be described in the general context of computer-executable instructions, such as program modules, executed by one or more computers or other devices. Generally, program modules include routines, programs, objects, components, data structures, and so on that perform particular tasks or implement particular abstract data types. Typically, the functionality of the program modules may be combined or distributed as desired in various embodiments.

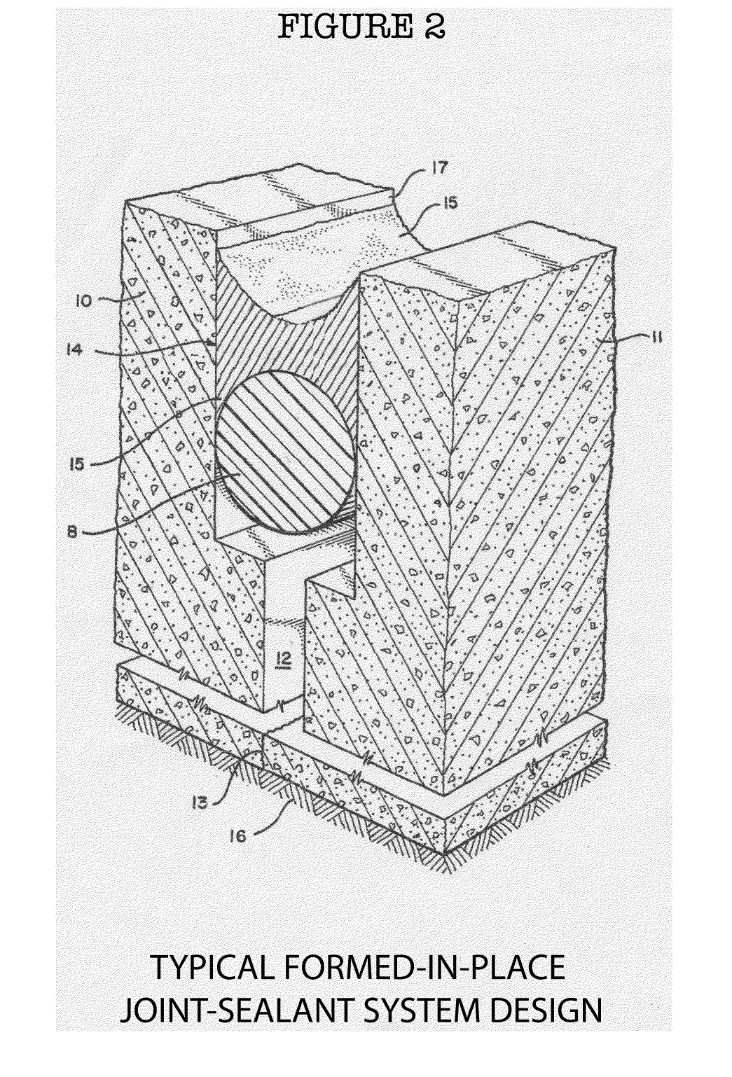

There are two distinct evaluation types that are performed by the joint inspection system, in some embodiments: 1) a joint-cleanliness evaluation that is associated with new construction or reconstruction of the joint, and 2) an in-place joint-sealant system evaluation occurring at some time after construction. The imaging approach for an in-place joint-sealant system evaluation is a modified version of the joint-cleanliness evaluation. In the joint-sealant system evaluation the traveler is modified to be above the joint-sealant system (or eliminated) and the (one or two) bore scope or miniature lenses of the camera system are placed above, and focused on the intersection of the sealant FIG. 2 (15) with the joint face FIG. 2 (17). The angle of the focus relative to the intersection and the lighting of the area being imaged will be determined by the researcher.

The research methodology can be used for construction joint cleanliness evaluations, or to develop a rapid, more efficient construction joint-cleanliness evaluation method.

In some embodiments, the construction joint-cleanliness research methodology uses the traveler FIG. 3 (A), set-up for indirect imaging FIG. 3 (B) using the visible light spectrum. The choice of the HD video camera system may be between a bore-scope and a miniature lens, to be determined by the researcher. It is expected that each joint face FIG. 2 (17) will be imaged in a single pass. In some embodiments, this will use two imaging (camera) assemblies, one camera for each joint wall. The imagery is integrated with location data (e.g., from 0.5+ centimeter accuracy GPS receiver FIG. 3 (C)) (similar to Magellan's dual-frequency LRK TM kinematic processing technology) mounted on the carriage FIG. 3 (G). If a more accurate GPS location system is required, one is available. The GPS receiver and the imaging systems will send their output to an onboard, or remote, computing device FIG. 3 (D) that integrates the data streams and stores the integrated data in a data store FIG. 3 (E). If desired, the data can be displayed in real-time to the equipment operator.

Carriage guidance, in some embodiments, is by two vertical pins (one each attached to the front and rear differentials of the carriages drive system) extending down into the joint channel FIG. 2 (14). Utilizing pin guidance minimizes the chances of operator guidance error in controlling the carriage. The operator can have radio control over the carriage speed. The carriage in one embodiment of the research version is a radio controlled, four wheel drive, automotive model utilizing the chassis, stabilized-suspension system and tires of an “Axial” SCX10 Deadbolt 1/10th scale electric 4 wheel-drive model that has been modified, with a slower speed motor and reduction gear, which permits the model to move at a speed of one to six feet per minute.

The actual speed used during data acquisition will be determined by the researcher and is primarily a function of the conditions in the field, although the relationship between speed and adequacy of the HD video may be a factor.

The joint inspection system has multiple research related data advantages. Field data collection is faster and safer. The data is verifiable, (repeat evaluations of the recorded data will produce equivalent results) and are comparable with future data. The actual data evaluation by the researcher occurs in the office and not, as currently done, in the field. Images of specific areas of concern can be enlarged and evaluated in-depth. If there is a question as to what is being observed, the area of concern can be reevaluated by others. None of this is possible with the current methodology. In addition, the data can be used administratively for specification development, and compliance.

From a field operations perspective, the in-place joint-sealant system evaluation has multiple advantages over current field data collection procedures. One is traffic safety considerations. Typically, evaluation of a test site requires the researcher to evaluate the joint on his hands and knees utilizing a ruler that extends over the length the transverse joint (typically 12 feet per traffic lane and 10 feet per shoulder lane). A similar approach is used to evaluate the longitudinal joints. With the joint inspection system, the operator only has to approach the traveled way to remove the system embodiment from the joint. His exposure in comparison to the current procedures is nominal. Wi-Fi to an onsite laptop permits the researcher to evaluate the quality of the data being recorded in real-time, so that the inspector can stop and reverse for quick reevaluation of suspect sections. The researcher can also mark areas appropriate for field sampling and have a sample taken (either then or later). Sampling while the inspection scope is still in place on the sampled joint (or on the test site) may have positive economic benefits by preventing repeat visits or other extra effort,

A trained researcher can evaluate one to four feet of joint per minute depending on the level of failures. On average, it takes from ten to fifteen minutes for a researcher, using a voice recorder, to evaluate a 12-foot transverse joint and set-up on the next transverse joint. Embodiments of the joint inspection system can travel at a speed of two feet per minute or more. At this rate, the operator can accumulate data for later office evaluation/review/comparison and immediate on-site evaluation for at least twice the number of joints that can be done with current methods. These evaluations usually occur on trafficked pavements. Usually the lane(s) to be evaluated are coned off from the traveled way and one or more flaggers are used to direct traffic. In most circumstances, the traffic in the traveled way is moving at the normal highway speeds for that particular section (if the roadway section being evaluated is two-lane opposed traffic then the speed and flagging conditions will vary). The closer the researcher is to the traffic lane the more vulnerable he is. His concentration has to be on the condition of the joint.

Typically there are at least two researchers and one or more flagger flaggers, evaluating a joint-sealant system test site; one researcher evaluating the joint, the other setting-up the ruler for the next joint evaluation, taking photography of specific points of interest, sampling as required, and documenting the test site. Using the joint inspection system, coning set-up and takedown will be equivalent. However, only one flagger and one operator are required. Utilization of the research version of the system for research related in-place joint-sealant system evaluations will provide significant savings in personnel costs (about 25% of current cost) and a major reduction in traffic exposure of personnel and potential construction zone type user accidents.

Construction site joint-cleanliness evaluations have different safety considerations than field joint-sealant system evaluations on traveled highways. Traffic related safety exposure of inspectors is typically nominal in construction site joint cleanliness evaluations. Specific construction site hazards vary with the site. From an administrative perspective the initial time cost of the inspector doing the joint-cleanliness evaluation with the research version of the system will be greater than the current inspection approach, possibly substantially greater if multiple inspections are required. Multiple inspections would be required if inadequate cleaning or the presence of moisture were detected. Moisture (condensation) is rarely detected today and the extent of moisture related adhesion failures is significantly underestimated. Offsetting the higher inspection costs are: significantly improved joint-sealant system performance, specification enforcement penalties on the contractor, and improved project documentation. Current field inspection utilizes minimal, non-repeatable, inadequate procedures (e.g., finger/black cloth inserted into the joint and checked for visible issues).

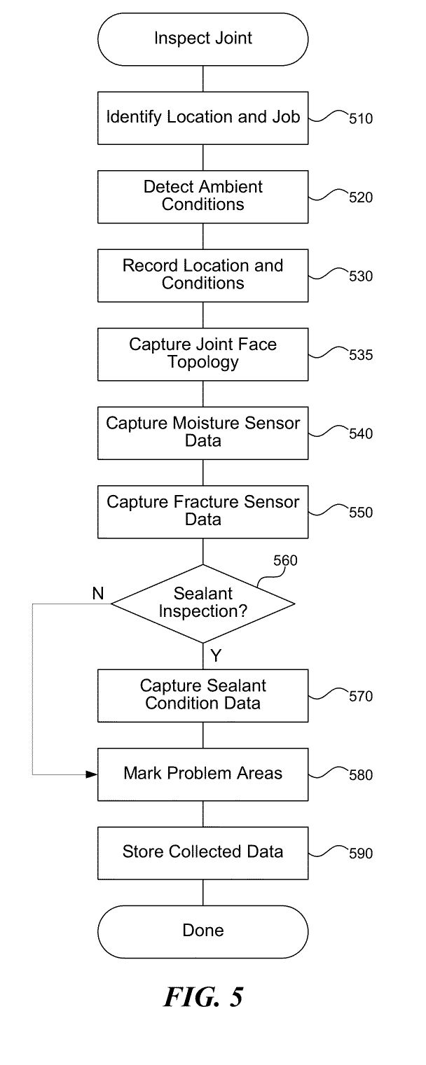

FIG. 5 is a flow diagram that illustrates processing of the joint inspection system to inspect a concrete joint, in one embodiment. Beginning in block 510, the system identifies a location and job where an operator has deployed the system to perform an inspection of a concrete joint. The location information may include a detected GPS location as well as location information entered by the operator, such as a numerical identification of a section of roadway, the roadway name, and so forth. The job information may include information describing the purpose of the inspection, the type of inspection, a contract associated with the inspection, and so on. The system may receive this information from an operator or may access other electronic systems to automatically determine this information.

Continuing in block 520, the system detects one or more ambient conditions relevant to the joint inspection for association with data collected during the joint inspection. Ambient conditions may include conditions such as humidity, weather forecast, dew point, temperature, time of day, and any other factor that could inform someone later analyzing the joint inspection data of one or more reasons why the data is what it is. For example, high humidity may help to explain condensation found within a joint during the joint inspection.

Continuing in block 530, the system records the identified location and ambient conditions for association with all of the data collected during the joint inspection. The system may use one or more on-board or remote data storage devices or a combination of these to store data collected and to identify the data with the location and conditions under which the data is being collected. Upon later retrieval of the data, researchers, operators, or others can access not only the inspection data itself but also useful information about the conditions surrounding the collection of the data.

Continuing in block 535, the system captures a topology of the joint face. The system may use one or more cameras, lasers, or other techniques for capturing the topology of the joint face. This visible or topological information is evaluated to determine the presence of contaminants on the surface that would interfere with the adhesion of the sealant or in the case of preformed sealants the bonding adhesive. Visible data is information intense and may require a judgmental evaluation based on reference images or may be automatically evaluated. Reference images can demonstrate various degrees of contamination and indicate whether the surface is sufficiently contaminant free to continue with sealing. In some embodiments, the joint sealant system captures visible data with a full depth angled mirror to capture the full depth of the sealant system to be placed. The mirror may also be angled to capture the frontal image of that joint face. The sealant system includes all of the area of the joint face shown in FIG. 2 from the base of the backer rod to the pavement surface. Existing endoscopes with a typical mirror attachment may be able to capture a small portion of the frontal image but would require significant manipulation to acquire the same visible information provided by the full depth angled mirror described herein. Also, the typical endoscope can provide a perspective view down the length of the joint with a larger but limited angle view of the surface. Neither view, frontal or angled, provides the level of information obtainable with the angled, full-depth mirror system described herein. The joint sealant system described herein provides repeatable, numeric data that can be statistically evaluated within a computer to provide significantly more rapid and consistent evaluation. The system is able to identify condensation and other issues on the joint face that would not be caught by existing methods. The system also permits inspection immediately before sealant installation, so that the conditions immediately prior to installation are known and documented for later evaluation, should the sealant fail.

While moving along the length of a pavement joint, the system performs the following steps. Continuing in block 540, the system captures moisture sensor data that identifies moisture information within the joint. The moisture information may be captured from one or more hardware sensors, such as a humidity sensor, as well as may be inferred or analyzed from digital imagery captured within the joint, such as through the analysis of a digital image to identify condensation. The system records the moisture information for later analysis and may display moisture readings to the operator during the inspection. This can alert the operator to request more detailed collection of data at any areas of the joint that differ substantially in sensor readings from the rest or that differ substantially from past data collected at the same location.

Continuing in block 550, the system captures fracture sensor data that identifies micro-fractures within or on the surface of the joint. Fractures may indicate improper construction sawing, freeze/thaw cycles, debris interference with the joint, or other conditions that may signal immediate problems as well as potential future problems such as spalling, poor joint sealant adhesion, and the like. The system may identify fractures using various spectra of light, vibration-based sensing, digital image analysis, microscopic observation, or other methods of capturing data and identifying fracturing in a surface. The system may be able to detect fractures much smaller than those that can be perceived by the human eye or with any kind of manual analysis, and thus can alert an operator that remediation is needed that the operator alone could not have detected.

Continuing in decision block 560, if the system is performing an inspection of already applied sealant, then the system continues at block 570, else the system jumps to block 580. Continuing in block 570, the system captures sealant condition data that identifies one or more conditions of existing sealant in the joint. Once sealing of a joint has taken place, an operator may perform routine inspections of the sealant within the joint to determine how the sealant is performing and whether the joint is facing any potential failure conditions. For example, the sealant condition may indicate that the sealant is drying out and cracking, has failed to adhere to the joint surface, or other conditions that may indicate future failure of the sealant to protect the joint from water-related, debris-related, or other damage.

Continuing in block 580, the system optionally places a physical mark upon one or more areas of the joint with identified anomalies that is visible to another party that will perform remediation of the anomalies. For example, the system may apply a color-coded paint mark to the top of the joint in areas where particular detected defects are detected, such as poor adhesion, presence of debris, presence of moisture, and the like.

Continuing in block 590, the system stores the collected sensor data and recorded location and conditions for subsequent use by one or more analyzers of the data. The system may store data both locally with the sensor apparatus as well as remotely, such as in a central data storage facility where the data can be shared and analyzed by multiple interested parties, such as a jurisdiction responsible for maintenance of the roadway, researchers, sealant manufacturers, and others. After block 590, these steps conclude.

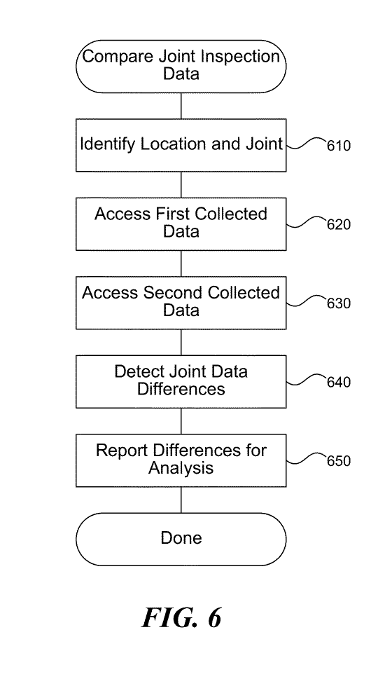

FIG. 6 is a flow diagram that illustrates processing of the joint inspection system to compare joint inspection data from two or more subsequent inspections, in one embodiment. Beginning in block 610, the system identifies a specific location and concrete joint for which two data collection instances will be compared. The system may provide a database or other data store from which data collection instances can be selected by a user of the system, such as a researcher analyzing joint sealant performance over time. The system may provide an interface for selecting, searching, and filtering data sets so that a particular joint location and instance can be selected by the user. In some cases, a past data set may be selected and the second set to compare with may come in real-time from a sensor or system collecting new, present data.

Continuing in block 620, the system accesses a first previously recorded and collected joint sensor data instance. The joint sensor data instance includes at least location and time of capture information and may include one or more of digital imagery captured at the location, moisture sensor data, fracture sensor data, sealant sensor data, and other types of data. Digital imagery may include one or more digital images or video capturing one or more angles of a joint. Through the use of mirrors and other optical techniques, the digital images may capture more than one angle in a particular image or the data may contain multiple images for the different possible angles (e.g., surface of joint, left face inside joint, right face inside joint, bottom of joint, and so on).

Continuing in block 630, the system accesses a second previously recorded and collected joint sensor data instance. The second data instance is captured later than the first data instance, which could be a long period, such as a period of months or years. In some cases, both the first and second instances will be of an already installed, sealed joint, while in other cases the first instance may be construction-time, pre-sealing data, while the second instance is post-construction, sealed joint data. In other cases, the later data instance may be post-failure data. Although shown as a comparison of two data instances, the method herein can be applied to compare any number of data instances, such as all of the data instances collected for a particular joint throughout the joints lifetime.

Continuing in block 640, the system detects one or more differences between the first and second data instances to identify performance of the concrete joint over time with a level of specificity greater than what can be achieved by manual human observation. The system allows for detection of conditions at such a micro level or at such an early stage that a human observer would not see with the naked eye that any failure or anomaly was occurring, but the system can detect these issues in a repeatable and comparable manner. In addition, the system does not experience human fatigue, boredom, sloppiness, inconsistency, or other issues that might lead to failure to catch particular problems within the joint and evidenced by comparing the joint data instances.

Continuing in block 650, the system reports the one or more detected differences to an operator to schedule remediation of one or more potential causes of joint failure. The system may provide a variety of types of reports, such as on-screen display to the operator, printed reports, emailed reports, web pages displaying the report data, mobile application for displaying roadway reports, and so forth. The system may store or-re-generate reports so that the operator, researchers, and others can review reports again over time. After block 650, these steps conclude.

The above steps detail a research method of the joint inspection system. Construction joint inspection is a sampling procedure that involves repeatedly inspecting, making a go/no-go decision, and re-inspecting until the conditions of the joint are correct. Then sealant is installed, potentially post-inspected, and a document is generated to give a contractor/owner verification of the validity of the installation.

In some embodiments, the joint inspection system includes a drag-operated variation. A bore-scope similar to a “Teslong” digital video recording endoscope (endoscope) can be inserted into and dragged along the joint channel (e.g., with viewing rearward). However, the bore-scope camera-head would be rapidly abraded by the concrete and rendered non-functional. A modified traveler FIG. 3 (A) independent of the carriage FIG. 3 (G) would both support and protect the camera head FIG. 3 (B′), or it can be supported by an unpowered version of the carriage FIG. 3 (G) with fewer components.

In the drag variation, the traveler is connected directly to the endoscope by the fiber-optic cable, which functions both to transmit the images and to pull the traveler along the joint channel. In this variation, the traveler can be configured in at least two alternatives. First, it can be suspended from a small-wheeled carriage with guidance (pins), tires, and suspension similar to those on the “Axial” SCX10 Deadbolt but without the motor. In this version, the abrasion resistant construction used in the research version would be adequate. With the suspended traveler, it is possible to use the drag variation with the bore scope lens aimed forward. A battery powered low-pressure pump FIG. 3 (J) can be mounted on the carriage to keep the lens clear. Second, the unsuspended version of the traveler can be built to withstand the abrasion effects of being dragged along the bottom of the joint channel. There is an operational problem with this version. It is more likely to create dust than the suspended version. The dust in the air may be insufficient to create a viewing obstacle, but it will probably tend to fog the lens.

With the drag variation, it may be possible for the inspector to identify both joint face contamination and moisture on the hand held view screen of the endoscope. If this can be done, the inspector can manually mark the pavement surface next to the contaminated areas (e.g., with paint spray, chalk). This system can also allow the inspector or others to review contaminated areas and make adjustment or corrections in the cleaning process. The endoscope has an onboard memory and some manufacturers may have both GPS and microphone capabilities that would allow location information and user comments to be added to the visuals. This approach is a very significant improvement over current methods. Most importantly from a practical standpoint, the drag variation can be implemented immediately. The ability of the drag variation to adequately show specific conditions existing on the joint may depend on certain factors. If done in conjunction with the traveler mirror system, the drag variation imaging would be acceptable for point or short-section inspections.

In some embodiments, the joint inspection system includes a research construction cleanliness variation. The research version can be trimmed down for joint cleanliness evaluations, and possibly for identification of moisture on the joint face. If the traveler FIG. 3 (A) uses the bore scope in the channel FIG. 3 (B′), it can mimic the drag variation operations and potentials. Although the research version can be used for construction joint cleanliness evaluations, one of its purposes is to develop a simpler, more rapid evaluation methodology. This simpler version can include direct viewing of the joint channel with a bore scope FIG. 3 (B′), with or without the traveler FIG. 3 (A), or a mini-camera. The imaging system can be suspended from a radio controlled movable carriage mounted on either a fixed length or an extendable bar. If the bar is a fixed length, that length can be determined in the field. The bar can be attached to a hydraulic or electrical lift system that permits the bar to be rapidly raised or lowered and its imaging system positioned within the joint channel. The lift system can be attached to some type of vehicle to rapidly move from joint to joint.

The flexibility of the traveler concept permits development of other variations dependent upon need. For instance, laser evaluation of the topology of the channel surface may be desirable; or sonic evaluation of the integrity of the structure supporting the channel face may be possible. Any evaluation system requiring a mechanism to place a receptor (sender) in immediate proximity to the channel face can utilize the traveler concept.

There are several possibilities for rapid discrimination between a clean, diamond-sawn concrete joint channel and one contaminated with slurry or other debris. The diamond saw (or grinder type joint face cleaners) leaves a distinctive pattern on the sawn channel face. Patches of slurry or debris leave a different pattern. One way to rapidly distinguish between the two patterns is by using light to establish/create a gray scale.

From the foregoing, it will be appreciated that specific embodiments of the system have been described herein for purposes of illustration, but that various modifications may be made without deviating from the spirit and scope of the invention. Accordingly, the invention is not limited except as by the appended claims.

I/We claim:

1. A computer-implemented method to inspect a concrete joint, the method comprising:
identifying a location and job where an operator has deployed a joint inspection system to perform an inspection of a concrete joint;
detecting one or more ambient conditions relevant to the joint inspection for association with data collected during the joint inspection;
recording the identified location and ambient conditions for association with all of the data collected during the joint inspection;
while moving along the length of a pavement joint:
capturing moisture sensor data that identifies moisture information within the joint;
capturing fracture sensor data that identifies micro-fractures within or on the surface of the joint; and
storing the collected sensor data and recorded location and conditions for subsequent use by one or more analyzers of the data,
wherein the preceding steps are performed by at least one processor.

2. The method of claim 1 wherein identifying the location and job comprises invoking global positioning system (GPS) hardware to determine a present location of the system.

3. The method of claim 1 wherein identifying the location and job comprises identifying a type of the inspection selected from the group consisting of a joint cleanliness inspection and an in-place sealant evaluation.

4. The method of claim 1 wherein detecting one or more ambient conditions comprises detecting at least one of humidity, weather forecast, dew point, temperature, and time of day.

5. The method of claim 1 wherein recording comprises storing collected data to one or more on-board or remote data storage devices and identifying the data with the location and conditions under which the data is being collected.

6. The method of claim 1 wherein capturing moisture sensor data comprises invoking one or more hardware sensors including a humidity sensor.

7. The method of claim 1 wherein capturing moisture sensor data comprises collecting digital imagery and analyzing the digital imagery for signs of moisture.

8. The method of claim 1 wherein capturing moisture sensor data comprises displaying moisture readings to the operator during the inspection.

9. The method of claim 1 wherein capturing fracture sensor data comprises detecting at least one of improper construction sawing, freeze/thaw cycles, and debris interference with the joint.

10. The method of claim 1 wherein capturing fracture sensor data comprises using at least one of lasers, light spectrum readings, or vibration-based sensing to detect micro-fractures within the concrete on either side of the joint.

11. The method of claim 1 further comprising if the system is performing an inspection of already applied sealant capturing sealant condition data that identifies one or more conditions of existing sealant in the joint.

12. The method of claim 1 further comprising placing a physical mark upon one or more areas of the joint with identified anomalies that is visible to another party that will perform remediation of the anomalies.

13. A computer system for performing repeatable and comparable inspections of concrete joints, the system comprising:
a processor and memory configured to execute software instructions embodied within the following components;
a user interface component that interacts with an operator of the system to receive input and provide output related to a joint inspection;
a location-recording component that identifies and captures location information of a location where the joint inspection is taking place;
an inspection component that manages the joint inspection and coordinates the capturing of sensor data and the recording and storage of sensor data for subsequent analysis;
a moisture sensor component that senses moisture information within a joint being inspected;
a fracture sensor component that detects fractures within a joint that may affect the performance of the joint;
a sealant sensor component that is used to inspect previously installed sealant and to assess a present condition of the sealant;
a data collection and storage component that receives and stores sensor data as well as metadata describing the location at which sensor data was captured; and
a data comparison component that provides analysis of multiple captures of joint data separated in time to identify changes in conditions of the joint between a first inspection and a second inspection of the joint.

14. The system of claim 13 wherein the user interface component displays real-time inspection results and comparison data with a past inspection of the same joint to the operator.

15. The system of claim 13 wherein the location recording component captures environmental information and associates this information with the data collected during the inspection, so that later analysis can factor in any relevant environmental factors that may be causes of anomalies or other joint conditions.

16. The system of claim 13 further comprising a marking component that provides a facility to physically mark the joint at locations where one or more joint issues are detected by the system.

17. The system of claim 13 wherein the data comparison component outputs a report to the operator detailing changes in the joint since the last inspection.

18. The system of claim 13 wherein the data comparison component provides predictive output including a prediction of likely types of failure of the joint based on the observed conditions and the rate of change of those conditions between the first and second inspections.

19. A computer-readable storage medium comprising instructions for controlling a computer system to compare joint inspection data from two or more subsequent inspections, wherein the instructions, upon execution, cause a processor to perform actions comprising:
identifying a specific location and concrete joint for which two data collection instances will be compared;
accessing a first previously recorded and collected joint sensor data instance;
accessing a second previously recorded and collected joint sensor data instance;
detecting one or more differences between the first and second data instances to identify performance of the concrete joint over time with a level of specificity greater than what can be achieved by manual human observation; and
reporting the one or more detected differences to an operator to schedule remediation of one or more potential causes of joint failure.

20. The medium of claim 19 wherein each joint sensor data instance includes at least location and time of capture information and at least one of digital imagery captured at the location, moisture sensor data, fracture sensor data, and sealant sensor data.
